(19) United States
(12) Patent Application Publication
(10) Pub. No.: US 2024/0033395 A1
(43) Pub. Date: Feb. 1, 2024

(54) AIR STERILISATION UNIT

(71) Applicant: ILIMEX LIMITED, Ballycastle (GB)

(72) Inventor: Richard MCCAULEY, Ballycastle (GB)

(21) Appl. No.: 18/267,966

(22) PCT Filed: Dec. 7, 2021
(86) PCT No.: PCT/EP2021/084655

§ 371 Date: Jun. 16, 2023

(30) Foreign Application Priority Data
Dec. 18, 2020 (GB) 2020161.2

(51) Int. Cl. A61L 9/20; B01D 46/12; B01D 46/00

(52) U.S. Cl. CPC A61L 9/205; B01D 46/12; B01D 46/0005; B01D 46/0027; A61L 2209/14

(57) ABSTRACT

An air sterilisation unit comprising a housing providing an air inlet and an air outlet and containing a filter module located between the air inlet and the air outlet, a UV-C treatment chamber located between the air inlet and the air outlet and containing at least one UV-C radiation source, and a ventilation system located between the air inlet, through the filter module and the UV-C treatment chamber and out of the housing through the air outlet, wherein the UV-C treatment chamber comprises an inlet end, an outlet end and a wall there between and the inlet end is closed with a first expanded polytetrafluoroethylene filter, the outlet end is closed with a second expanded polytetrafluoroethylene and the wall is provided with an expanded polytetrafluoroethylene liner, such that the UV-C radiation source is surrounded with expanded polytetrafluoroethylene for reflection of UV-C radiation within the chamber for destruction of pathogens in the air.

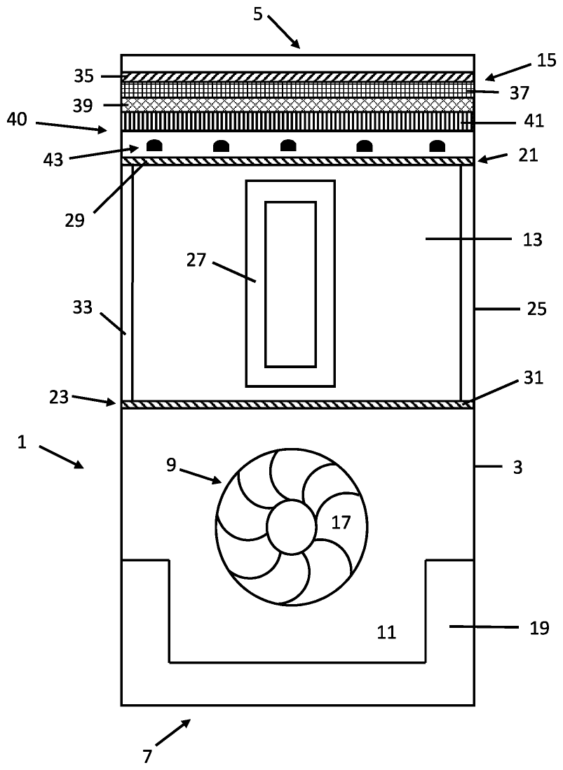

The present invention relates to an air sterilisation unit, and in particular to an air sterilisation unit for the destruction of pathogens.

### BACKGROUND TO THE INVENTION

Many devices exist for treating air. Some of these simply filter the air, others provide filtering and sterilisation of the air. These devices have typically been used in places such as hospital areas, operating theatres, laboratories, clean room separation areas, air distribution systems, food packaging areas, etc. With the development of the Covid-19 pandemic, air treatment devices are being deployed more and more in any place where people gather, such as hotels and restaurants, etc.

In all of these places, there is a need to avoid or minimise the presence of pathogens, such as bacteria, viruses, fungi, and other air contaminants, such as dust, smoke, pollen, volatile organic compounds, etc. For this purpose, air treatment devices have been provided which, to varying degrees, remove pathogens and other contaminants from air. In these devices, it is known to pass air through one or more filters and into a chamber having one or more UV-C radiation lamps. The filters act to remove air contaminants and the UV-C radiation acts to destroy pathogens in the air.

However, problems exist with known air treatment devices. For example, due to the configuration and composition of the filters and UV-C radiation chamber, the air can receive insufficient UV-C radiation to destroy an adequate number of airborne pathogens. This results in a decreased efficacy of the devices and limits deployment in places where high pathogen removal is required. The significance of this issue has increased sharply due to the Covid-19 pandemic, as all places of deployment of air treatment devices seek to provide a high pathogen kill level.

### SUMMARY OF THE INVENTION

According to the first aspect of the invention there is provided an air sterilisation unit comprising:
a housing providing an air inlet and an air outlet and containing:
a filter module located between the air inlet and the air outlet,
a UV-C treatment chamber located between the air inlet and the air outlet and containing at least one UV-C radiation source, and
a ventilation system located between the air inlet and the air outlet, configured to cause air flow into the housing through the air inlet, through the filter module and the UV-C treatment chamber and out of the housing through the air outlet,
wherein the UV-C treatment chamber comprises an inlet end, an outlet end and a wall there between and the inlet end is closed with a first expanded polytetrafluoroethylene filter, the outlet end is closed with a second expanded polytetrafluoroethylene and the wall is provided with an expanded polytetrafluoroethylene liner, such that the UV-C radiation source is surrounded with expanded polytetrafluoroethylene for reflection of UV-C radiation within the chamber for destruction of pathogens in the air.

The expanded polytetrafluoroethylene (ePTFE) surrounding the UV-C radiation source in the UV-C treatment chamber increases the reflection of the UV-C radiation within the chamber and increases the UV-C radiation fluence within the chamber. The average UV-C radiation fluence within the UV-C treatment chamber is preferably greater than 30000 μJ/cm². The liner may comprise ePTFE soft sheet. The liner may have a minimum thickness of 2 mm.

The sterilisation unit preferably achieves an 8 log reduction of pathogens in the air. In most circumstances, this provides substantially complete kill of pathogens in the air in one pass of the air through the UV-C treatment chamber of the unit.

The first and second ePTFE filters and the ePTFE liner preferably substantially contain the UV-C radiation within the UV-C treatment chamber. This protects other components of the air sterilisation unit from damage by the UV-C radiation.

The filter module may be located outside the UV-C treatment chamber. The filter module may be located between the air inlet of the housing and the inlet end of the UV-C treatment chamber. This separation of the filter module from the UV-C treatment chamber decreases UV-C radiation absorption which occurs when filters are located within the chamber.

The filter module may comprise a plurality of filters. The plurality of filters may comprise at least one mesh filter. The mesh filter may trap contaminants in the air of a size in the range of 3-10 microns. The contaminants may comprise any of hair, skin, large dust particles, large dirt particles. The mesh filter may be removable from the filter module. The mesh filter may be washable.

The plurality of filters may comprise at least one high efficiency particulate air (HEPA) filter. The HEPA filter may be doped with silver ions. The HEPA filter may trap contaminants in the air of a size up to 0.3 microns. The contaminants may comprise any of pollen, smoke, dust, dirt.

The plurality of filters may comprise at least one activated carbon filter. The activated carbon filter may trap contaminants comprising any of volatile organic compounds (VOCs), smoke, formaldehydes, smells, benzene, toluene, xylene, chlorinated compounds.

It will be appreciated that the plurality of filters may comprise other types of filters, such as electrostatic filters.

The filter module may comprise a UV-A treatment chamber. The UV-A treatment chamber may comprise at least one photocatalytic (POC) filter and one or more UV-A radiation sources for activation of the POC filter.

The POC filter may comprise a block of aluminium having a honeycomb-shaped interior provided with a POC coating. The POC coating may comprise a titanium dioxide (TiO₂) coating. On activation, the POC coating destroys air pollutants and pathogens which contact the filter.

The or each UV-A radiation source may comprise a UV-A light emitting diode (LED). The or each UV-A LED may provide a beam of UV-A radiation. The or each beam of UV-A radiation may be focused by a lens to be incident on the POC filter. This increases penetration of UV-A radiation into the POC filter. The or each UV-A radiation source may provide a total UV-A radiation power of approximately 30 W. UV-A radiation has a longer wavelength than UV-C radiation. Using UV-A radiation to irradiate the POC filter provides better penetration and therefore activation of the filter.

The ventilation system may provide a negative pressure within the housing to move air into the housing and to decrease escape of air from the housing before treatment thereof. The ventilation system may provide a predetermined rate of air flow through the housing. The ventilation system may comprise a fan.

The housing may further contain a dispersion chamber. The dispersion chamber may be located adjacent to the air outlet. The dispersion chamber may be sized to provide an area of the air outlet which is at least twice an area of the air inlet. This reduces operating noise of the system and outlet air draughts. The dispersion chamber may contain a porous sound-deadening material at least partially lining the dispersion chamber. The sound-deadening material may comprise a foam material.

At least one UV-C radiation source contained in the treatment chamber may comprise an electrodeless magnetic induction lamp. This has a long-life and the UV-C radiation output is less affected by air temperature variations.

According to a second aspect of the invention there is provided a method of sterilising air using the air sterilisation unit as described herein.

### BRIEF DESCRIPTION OF THE DRAWING

A preferred embodiment of the invention will now be described, by way of example only, with reference to the accompanying drawing which shows a cross sectional view from the front of an air sterilisation unit according to the invention.

### DETAILED DESCRIPTION

Referring to the drawing, a sterilisation unit 1 is provided, which draws air into the unit from the surrounding environment, treats the air and passes the treated air back into surrounding environment.

The sterilisation unit 1 comprises a housing 3. In this embodiment, the housing 3 is shown as having a rectangular shape, approximately 1250 mm long, 400 mm wide and 200 mm deep. It will be appreciated that the housing 3 can have any desired shape, for example cylindrical or triangular, and dimensions. A control panel (not shown) is provided on the outside of the housing 3 and a connecting cord (not shown) is provided for connecting the housing 3 to a mains power supply. It will be appreciated that the housing could alternatively or additionally comprise a battery.

The housing 3 provides an air inlet 5 at a top thereof and an air outlet 7 at a bottom thereof. It will be appreciated that the housing 3 could be oriented differently and the air inlet and outlet provided at sides of the housing.

The housing 3 contains a ventilation system 9, a dispersion chamber 11, a UV-C treatment chamber 13 and a filter module 15, each located between the air inlet 5 and the air outlet 7. The filter module 15 is adjacent the air inlet 5 at the top of the housing 3, the UV-C treatment chamber 13 is adjacent the filter module 15, the ventilation system 9 is adjacent the UV-C treatment chamber 13 and the dispersion chamber 11 is adjacent the outlet 7 at the bottom of the housing 3.

The ventilation system 9 comprises a fan 17 which is configured to cause air flow into the housing 3 through the air inlet 5, through the filter module 15 and the UV-C treatment chamber 13, into the dispersion chamber 11 and out of the housing 3 through the air outlet 7. The ventilation system 9 provides a negative pressure within the housing 3 to move air into the housing 3 and to decrease escape of air from the housing 3 before treatment thereof. The ventilation system 9 preferably provides a predetermined rate of air flow through the housing 3 of approximately 500 m³/hr.

The dispersion chamber 11 is located adjacent the air outlet 7 of the housing 3. The size of the dispersion chamber 11 reduces operating noise of the ventilation system 9 and reduces outlet air draughts. The dispersion chamber 11 contains a porous, sound-deadening, foam material 19 partially lining the dispersion chamber 11, as shown.

The UV-C treatment chamber 13 comprises an inlet end 21, an outlet end 23 and a wall 25 therebetween and contains a UV-C radiation source 27, which, in this embodiment, is an electrodeless magnetic induction lamp. The inlet end 21 of the UV-C treatment chamber 13 is closed with a first ePTFE filter 29. The outlet end 23 is closed with a second ePTFE filter 31. The wall 25 is provided with an ePTFE liner 33. The liner comprises ePTFE soft sheet and has a minimum thickness of 2 mm. The UV-C radiation source 27 is thus surrounded with ePTFE for reflection of UV-C radiation from the source 27 within the UV-C treatment chamber 13 for destruction of pathogens in air passing through the chamber 13.

The filter module 15 is located outside the UV-C treatment chamber 13, between the air inlet 5 of the housing 3 and the inlet end 21 of the UV-C treatment chamber 13. This separation of the filter module 15 from the UV-C treatment chamber 13 decreases UV-C radiation absorption which occurs when filters are located within the chamber 13.

The filter module 15 comprises a plurality of filters. The plurality of filters comprises a mesh filter 35. The mesh filter 35 traps contaminants in the air of a size in the range of 3-10 microns. The contaminants comprise contaminants such as hair, skin, large dust particles, large dirt particles. The mesh filter 35 is removable from the filter module 15, for example, to be washed.

The plurality of filters further comprises at least one high efficiency particulate air (HEPA) filter 37. The HEPA filter 37 is doped with silver ions. The HEPA filter 37 traps contaminants in the air of a size up to 0.3 microns. The contaminants may comprise any of pollen, smoke, dust, dirt.

The plurality of filters further comprises an activated carbon filter 39. This traps contaminants in the air such as volatile organic compounds (VOCs), formaldehydes, smells, etc.

The filter module 15 comprises a UV-A treatment chamber 40. The UV-A treatment chamber 40 comprises a POC filter 41 and a plurality of UV-A radiation sources 43 for activation of the POC filter. The POC filter 41 comprises a block of aluminium having a honeycomb-shaped interior provided with a POC coating of TiO₂. On activation, the POC coating destroys air pollutants and pathogens which contact the filter. The plurality of UV-A radiation sources 43 comprise a plurality of UV-A LEDs. The UV-A LEDs 43 provide a beam of UV-A radiation. The beams of UV-A radiation are each focused by a lens to be incident on the POC filter 41. This increases penetration of UV-A radiation into the POC filter 41. The UV-A LEDs 43 provide a total UV-A radiation power of approximately 30 W. UV-A radiation has a longer wavelength than UV-C radiation. Using UV-A radiation to irradiate the POC filter provides better penetration and therefore activation of the filter.

As air flows through the filter module 15, contaminants in the air are trapped by the plurality of filters and pathogens are destroyed in the UV-A treatment chamber. The air then passes into the UV-C treatment chamber 13, through the first ePTFE filter 29 at the inlet end 21 of the chamber 13.

The air is subjected to UV-C radiation from the UV-C radiation source 27, which destroys pathogens in the air. The ePTFE surrounding the UV-C radiation source 27 in the UV-C treatment chamber 13 increases the reflection of the UV-C radiation within the chamber 13 and increases the UV-C radiation fluence within the chamber 13. The average UV-C radiation fluence within the UV-C treatment chamber 13 is preferably greater than 30000 μJ/cm². The sterilisation unit 1 achieves an 8 log reduction of pathogens in the air, when tested with virus MS2, which, in most circumstances, provides a substantially complete kill of pathogens in the air in one pass of the air through the UV-C treatment chamber 13 of the unit.

The first and second ePTFE filters 29, 31 and the ePTFE liner 33 also substantially contain the UV-C radiation within the UV-C treatment chamber 13. This protects other components of the air sterilisation unit 1 from damage by the UV-C radiation.

It is to be understood that the invention is not limited to the specific details described herein which are given by way of example only and that various modifications and alterations are possible without departing from the scope of the invention as defined in the appended claims.

1. An air sterilisation unit comprising:
a housing providing an air inlet and an air outlet and containing:
a filter module located between the air inlet and the air outlet,
a UV-C treatment chamber located between the air inlet and the air outlet and containing at least one UV-C radiation source, and
a ventilation system located between the air inlet and the air outlet, configured to cause air flow into the housing through the air inlet, through the filter module and the UV-C treatment chamber and out of the housing through the air outlet,
wherein the UV-C treatment chamber comprises an inlet end, an outlet end and a wall therebetween and the inlet end is closed with a first expanded polytetrafluoroethylene filter, the outlet end is closed with a second expanded polytetrafluoroethylene and the wall is provided with an expanded polytetrafluoroethylene liner, such that the UV-C radiation source is surrounded with expanded polytetrafluoroethylene for reflection of UV-C radiation within the chamber for destruction of pathogens in the air.

2. An air sterilisation unit according to claim 1 in which the average UV-C radiation fluence within the UV-C treatment chamber is greater than 30000 μJ/cm².

3. An air sterilisation unit according to claim 1 in which the filter module is located outside the UV-C treatment chamber.

4. An air sterilisation unit according to claim 3 in which the filter module is located between the air inlet of the housing and the inlet end of the UV-C treatment chamber.

5. An air sterilisation unit according to in which the filter module comprises a plurality of filters.

6. An air sterilisation unit according to claim 5 in which the plurality of filters comprises at least one mesh filter, which traps contaminants in the air of a size in the range of 3-10 microns.

7. An air sterilisation unit according to claim 5 in which the plurality of filters comprises at least one high efficiency particulate air filter, doped with silver ions, which traps contaminants in the air of a size up to 0.3 microns.

8. An air sterilisation unit according to claim 5 in which the plurality of filters comprises at least one activated carbon filter.

9. An air sterilisation unit according to claim 1 in which the filter module comprises a UV-A treatment chamber.

10. An air sterilisation unit according to claim 9 in which the UV-A treatment chamber comprises at least one photocatalytic (POC) filter and one or more UV-A radiation sources for activation of the POC filter.

11. An air sterilisation unit according to claim 10 in which the POC filter comprises a block of aluminium having a honeycomb-shaped interior provided with a POC coating of titanium dioxide.

12. An air sterilisation unit according to claim 10 in which the or each UV-A radiation source comprises a UV-A light emitting diode (LED).

13. An air sterilisation unit according to claim 12 in which the or each UV-A LED provides a beam of UV-A radiation which is focused by a lens to be incident on the POC filter.

14. An air sterilisation unit according to claim in which the or each UV-A LED provides a total UV-A radiation power of approximately 30 W.

15. An air sterilisation unit configured to achieve an 8 log reduction of pathogens in the air.

16. A method of sterilising air using the air sterilisation unit of claim 1.
